(19) United States
(12) Patent Application Publication
(10) Pub. No.: US 2006/0074287 A1
(43) Pub. Date: Apr. 6, 2006

(54) Systems, methods and apparatus for dual mammography image detection

(75) Inventors: David C. Neumann, Milwaukee, WI (US); Habib Vafi, Brookfield, WI (US)

Correspondence Address: JAMES D IVEY, 3025 TOTTERDELL STREET, OAKLAND, CA, 94611-1742, US

(73) Assignee: General Electric Company, Schenectady, NY (US)

(21) Appl. No.: 10/955,040

(22) Filed: Sep. 30, 2004

(51) Int. Cl. A61B 5/05

(52) U.S. Cl. 600/407

(57) ABSTRACT

Systems and methods are provided by which a mammography imaging system offers X-ray and ultrasound imaging that allows sharing of common hardware such as the computer and display. Small regions of interest are imaged with X-ray at higher image quality by using a second sensor with higher DQE than the full-field sensor can obtain. In some embodiments a specialized chamber is provided for securing the anatomy to a fixed location, ultrasound image data is collected along with ultrasound probe location and orientation data from sensors on a handheld probe from which data images can be viewed directly, or used to reconstruct tomographic images of any desired cross-section, or used for various “3-D” image visualization methods. An imaging schedule defined by location and orientation of an ultrasound probe is used to generate a three-dimensional ultrasound image.

### FIELD OF THE INVENTION

This invention relates generally to mammography imaging system, and more particularly to higher detective quantum efficiency images.

### BACKGROUND OF THE INVENTION

The use of X-ray technology for providing two-dimensional images of breast tissue for diagnosis of carcinoma or other abnormalities is in wide use. However, X-ray imaging of breast tissue has the inherent limitation in that a mammogram provides only a planar image of a three-dimensional object.

The detective quantum efficiency (“DQE”) of an image is the conventional measure of X-ray image quality. In simpler terms, the DQE is the resolution of the detector. DQE is constant across an image for a given detector and dose technique.

When a potential area of medical concern is indicated on a mammogram, the elevation or depth of the subject area within the two-dimensional image of the breast may be uncertain. Present digital X-ray imagers provide full field or nearly full field imaging. Alternate means or complementary imaging techniques and diagnosis such as biopsy may be needed to complete the diagnosis.

The main complementary imaging techniques to mammography are ultrasound and magnetic imaging resonance (MRI), which both have the advantage of not using ionizing radiation. The main advantages of ultrasound are that ultrasound imaging is relatively inexpensive and that ultrasound imaging works well also for dense breasts where mammography has difficulties. Ultrasound imaging also plays an important role as guidance for needle biopsy. A MRI system is useful for contrast enhanced dynamic study due to its sensitivity. However, much of the hardware, such as computer and display, are duplicated because the systems are built and sold separately.

For the reasons stated above, and for other reasons stated below which will become apparent to those skilled in the art upon reading and understanding the present specification, there is a need in the art for a means to examine detailed areas of a breast without a biopsy. There is also a need for improved complementary imaging techniques such as ultrasound that is capable of using existing mammography hardware and software. Further, there is a need in the art for a mammography system for generating tomosynthesis images from ultrasound data.

### BRIEF DESCRIPTION OF THE INVENTION

The above-mentioned shortcomings, disadvantages and problems are addressed herein, which will be understood by reading and studying the following specification.

In one aspect, a mammography system having an X-ray source, a breast compression plate, and a digital image receptor, the receptor comprising movement mechanism coupled to a first detector and a second detector for positioning said first and second detectors within said image receptor, a first detector operable to receive energy from said X-ray source and for providing roadmap data and X-ray source data, and, a second detector operable to receive X-ray source energy and for providing X-ray source data.

Another aspect, a mammography system having an X-ray source, a breast compression plate, and a digital image receptor, the receptor comprising a first detector receiving energy from said X-ray source and for providing X-ray source data, and an electrical connector capable of coupling at least one external device.

In yet another aspect, mammography system having an X-ray source, a breast compression plate, and a digital image receptor. The receptor having a detector receiving energy from said X-ray source and for providing X-ray source data. Additionally, the receptor has at least one ultrasonic detector and ultrasonic transmitter externally coupled to the receptor wherein ultrasonic measurements from the ultrasonic transmitter and ultrasonic detector are used in constructing an image of a patient's breast by the mammography system.

One aspect is to a mammography imaging system having an X-ray mammography imaging subsystem adapted to image a breast and an ultrasound mammography imaging subsystem adapted to image a breast. Further, the system recites a selector switch for selecting between the X-ray mammography imaging subsystem and ultrasound mammography imaging subsystem for imaging a breast. a display device configured to displaying at least one image obtained or stored by said device.

In another aspect, an apparatus for generating a three-dimensional ultrasound image describe comprising an ultrasound probe for generating ultrasound image data through spatial registration, a motion control system for movement of the probe in relation to the breast and for sensing the probe's position, the motion control system including a first-axis control, a second-axis control, a third-axis control, and a fourth axis control for movement of the probe. Further, a computer for generating the three-dimensional ultrasound image from the ultrasound image data and from information regarding the spatial registration.

In yet another aspect, an ultrasound system having an ultrasound probe; the ultrasound probe comprising: having a sensor capable of providing signals that represent position and orientation; and a device capable of correcting the position and orientation signals and capable of generating signals that represent the actual position and orientation of the ultrasound probe relative to an object.

Another aspect is method for generating a three-dimensional ultrasound image by the steps of storing an imaging schedule defined by location and orientation of an ultrasound probe; moving the ultrasound probe to a position that is defined by a location and an orientation; generating at least one ultrasound image with an indicia indicating location and orientation; storing the indicia that are indicative of location and orientation of the ultrasound image; storing the generate ultrasound image with an indicia indicating location and orientation; comparing the stored indicia and the stored imaging schedule; generating an indication of completion based on the comparison of the stored indicia and the stored imaging schedule; and, generating a three-dimensional ultrasound image from the store ultrasound image upon the indication of completion.

In yet another aspect, mammography method is performed by a mammography system having a breast shaped chamber for constraining a breast, the breast is positioned in a chamber; the ultrasound probe is moved to a desired location and ultrasound energy is applied to the breast; data is obtained from the reflected ultrasound energy; image representation is created from the obtained data; the image representation from the reflected ultrasound energy is stored for displaying.

### BRIEF DESCRIPTION OF THE DRAWINGS

FIG. 1 is a diagram illustrating a system-level overview of an embodiment for a mammography system;

FIG. 2 is a two detector receptacle for a mammography system;

FIG. 3 is a one detector receptacle and connector for a mammography system;

FIG. 4 is a diagram of a ultrasound probe for use in an implementation of mammography system;

FIG. 5 is a diagram illustrating a system-level overview of a mammography system that uses a chamber and ultrasound probe;

FIG. 6 is a diagram of an ultrasound probe having sensors and devices for determining position and orientation;

FIG. 7 is a mammography system employing an X-ray subsystem and ultrasound subsystem with a switch for selecting between the subsystems;

FIG. 8 is a mammography system with motion controller and position sensor;

FIG. 9 is a mammography system with first and second storage units with comparator; and

FIG. 10 is a block level diagram of data processing devices for controlling and sharing information from different locations.

### DETAILED DESCRIPTION OF THE INVENTION

In the following detailed description, reference is made to the accompanying drawings that form a part hereof, and in which is shown by way of illustration specific embodiments which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

The detailed description is divided into five sections. In the first section, a system level overview is described. In the second section, methods of embodiments are described. In the third section, the hardware and the operating environment in conjunction with which embodiments may be practiced are described. In the fourth section, particular implementations are described. Finally, in the fifth section, a conclusion of the detailed description is provided.

### System Level Overview

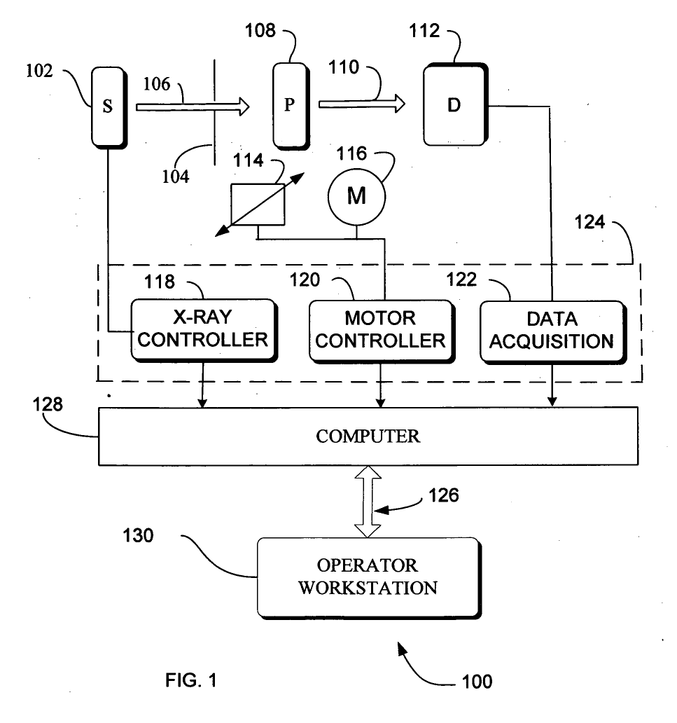

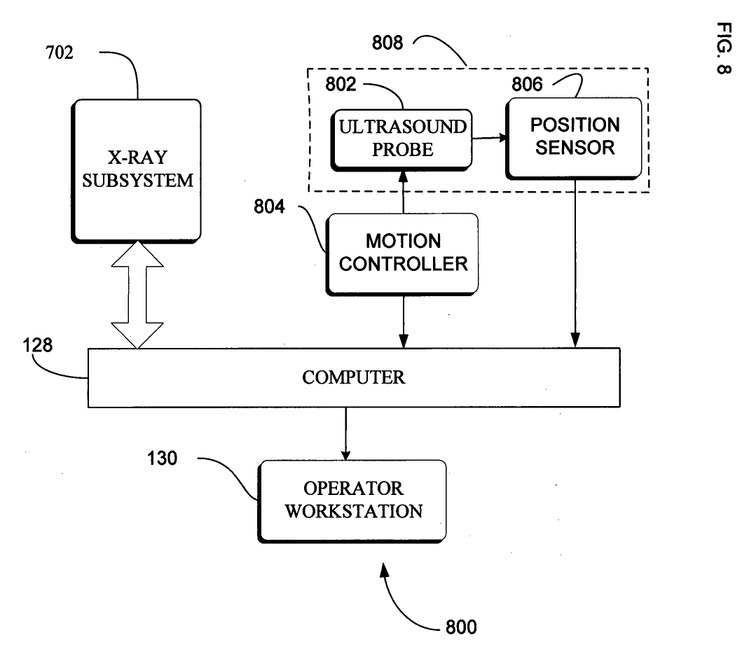

FIG. 1 is a block diagram that provides a system level overview. Embodiments are described as operating in a multi-processing, multi-threaded operating environment on a computer, such as computers 128 and 130 in FIG. 8.

FIG. 1 illustrates diagrammatically a mammography imaging system 100 for acquiring and processing tomography image data for full-field digital mammography (FFDM). In the illustrated embodiment, system 100 is a computed tomography (CT) system designed both to acquire original image data, and to process the image data for display and analysis. Alternative embodiments of system 100 can include a positron emission tomography (PET) mammography system, a nuclear medicine breast imaging system (scintimammography), a thermoacoustic tomography breast imaging system (TCT), an electrical impedance mammography system (EIT), near-infrared mammography systems (NIR), and X-ray tomosynthesis mammography systems (XR).

In FIG. 1, imaging system 100 includes a source of X-ray radiation 102 positioned adjacent to a collimator 104. In this arrangement, the source of X-ray radiation source 102 is typically an X-ray tube. Other modalities, however, possess different sources of imaging energy or radiation. For instance, modalities such as PET and nuclear medicine imaging utilize an injectable radionucleotide as a source 102, and source 102 encompasses such alternative sources of imaging energy or radiation which are utilized in tomography imaging systems. Imaging system 100 solves the need in the art for examining a detailed area of the breast without a biopsy.

Returning to the computed tomography of FIG. 1, the collimator 104 permits a stream of radiation 106 to pass into a region in which a subject, such as a human patient 108 is positioned. A portion of the radiation 110 passes through or around the subject and impacts a detector array, represented generally at reference numeral 112. In the full-filed digital mammography (FFDM) the detector can be of three types, which may be called indirect detection (charge collection), direct detection and direct photon counting. In the indirect detection systems (for instance photostimulable phosphors, CsI(Tl)-CCD and CsI(Tl)-αSi) light photons are emitted which in a second step leads to electric charges that will result in an electric signal in a photo detector. In direct detection (for instance αSe) the X-ray photons directly lead to charges (electron-hole pairs) and thus to an electric signal in a photoconductor. In both cases the electric signal produced is the result of interaction from typically hundreds of X-ray photons. The electric signal is digitized and represents the intensity level in a pixel. In direct photon counting techniques (for instance Si(B)) single photons are counted. In this case e.g. the number of photons directly represents the intensity level in a pixel.

Detector elements of the array produce electrical signals that represent the intensity of the incident X-ray beam. These signals are acquired and processed to reconstruct an image of the features within the subject. Source 102 is controlled by a system controller 124 which furnishes both power and control signals for CT examination sequences. Moreover, detector 112 is coupled to the system controller 124, which commands acquisition of the signals generated in the detector 112. The system controller 124 may also execute various signal processing and filtration functions, such as for initial adjustment of dynamic ranges, interleaving of digital image data, and so forth. In general, system controller 124 commands operation of the imaging system to execute examination protocols and to process acquired data. In the present context, system controller 124 also includes signal processing circuitry, typically based upon a general purpose or application-specific digital computer, associated memory circuitry for storing programs and routines executed by the computer, as well as configuration parameters and image data, interface circuits, and so forth.

In the arrangement illustrated in FIG. 1, system controller 124 is coupled to a linear positioning subsystem 114 and rotational subsystem 116. The rotational subsystem 116 enables the X-ray source 102, collimator 104 and the detector 112 to be rotated one or multiple turns around the region to be imaged. It should be noted that the rotational subsystem 116 may include a gantry suitably configured to receive the region to be imaged, such as a human breast in a CT mammography system. Thus, the system controller 124 may be utilized to operate the gantry.

The linear positioning subsystem 114 enables the region to be imaged to be displaced linearly, allowing images to be generated of particular areas of the patient 108.

Additionally, as will be appreciated by those skilled in the art, the source of radiation may be controlled by an X-ray controller 118 disposed within the system controller 124. Particularly, the X-ray controller 118 is configured to provide power and timing signals to the X-ray source 102. Those of ordinary skill in the art understand that the source 102, detector array 112, and X-ray controller 118 comprise suitable analog circuitry for performing their operations.

A motor controller 120 may be utilized to control the movement of the rotational subsystem 116 and the linear positioning subsystem 114. Further, the system controller 124 is also illustrated comprising a data acquisition system 122. In this arrangement, the detector 112 is coupled to the system controller 124, and more particularly to the data acquisition system 122. The data acquisition system 122 receives data collected by readout electronics of the detector 112. The data acquisition system 122 typically receives sampled analog signals from the detector 112 and coverts the data to digital signals for subsequent processing by a computer 128 through a data interchange device 126 such as a LAN, WAN, or Internet. The data acquisition 122 can be performed at the detector 122 level without departing from the concept of the invention.

The computer 128 is typically coupled to the system controller 124. The data collected by the data acquisition system 122 may be transmitted to the computer 128 and moreover, to a memory 1006, 1008, 1010. It should be understood that any type of memory to store a large amount of data may be utilized by such an exemplary system 100. Also the computer 128 is configured to receive commands and scanning parameters from an operator via an operator workstation 130 typically equipped with a keyboard and other input devices. An operator may control the system 100 via the input devices. Thus, the operator may observe the reconstructed image and other data relevant to the system from computer 128, initiate imaging, and so forth.

A display 1022 coupled to the operator workstation 130 or computer 128 may be utilized to observe the reconstructed image and to control imaging. For example, the General Electric SENOGRAPH® 2000D workstation. Additionally, the scanned image may also be printed on to a printer which may be coupled to the computer 128 and the operator workstation 130. Further, the operator workstation 130 may also be coupled to a picture archiving and communications system through appropriately programmed ports. It should be noted that picture archiving and communications system may be coupled to a remote system 1014, radiology department information system, and hospital information system or to an internal or external network, so that others at different locations may gain access to the image and to the image data as disclosed in FIG. 8.

It should be further noted that the computer 128 and operator workstation 130 may be coupled to other output devices which may include standard or special purpose computer monitors and associated processing circuitry. One or more operator workstations 130 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or, in an entirely different location, linked to the image acquisition system via one or more configurable networks, such as the Internet, virtual private networks, and so forth.

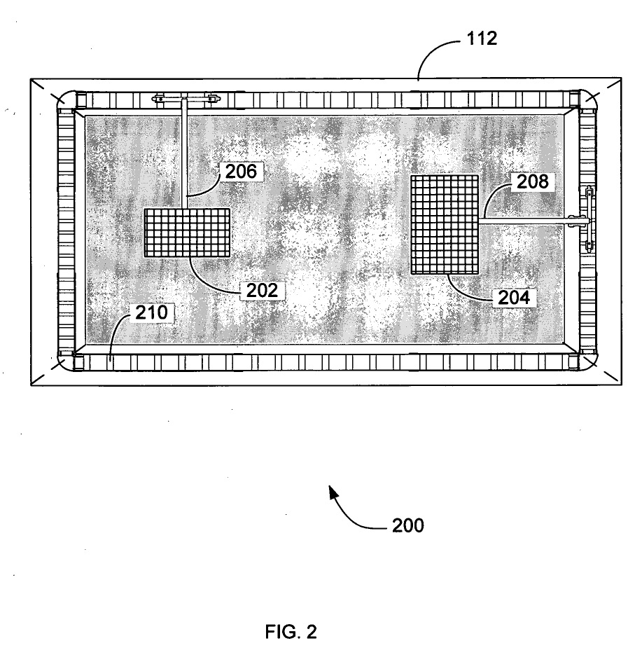

In FIG. 2, a dual sensor arrangement is shown for detector 112. Sensors 202 and 204 that form part of detector 112 are different sizes because a small image detection area with smaller pixel pitch or higher pixel density leads to higher detective quantum efficiency (DQE). The DQE is the performance of an imaging system and includes the noise and spatial resolution properties of the system as a function of the spatial frequency. In other words it is a measure of how efficient the detector can convert the information from the X-ray quanta to a useful signal to produce an image.

In FIG. 2, mechanism 206 and 208 is used to position the sensors 202, 204 at a desired position for conducting the imaging of the patient 108. Mechanisms 206 and 208 are individually coupled to a motion mechanism 210 for moving the sensors (202,204) to a desire location. The motion mechanism 210 can be a track or groove that facilitates movement within the receptacle of the detector 112. For example, sensor 204 can be initially position to measure an aspect of the breast. At the same time the mechanism is able to ascertain the position of sensor 204 if there is a desire to measure an aspect of the breast with a higher resolution. This position data is roadmap data that can be used to position sensor 202 to image a desired location using the higher DQE sensor.

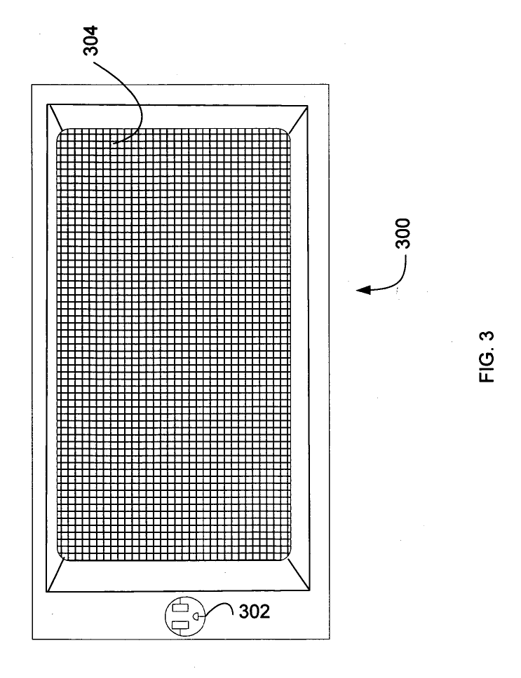

In FIG. 3, the detector 112 is augmented with a connector for an ultrasonic probe. The receptacle of detector 112 can be a standard receptacle with a connection for an ultrasound probe. This arrangement permits common image detection and display electronics to be shared by the detector 112 and the ultrasonic probe electrically coupled through connector 302. Imaging system 300 solves the need in the art for complementary imaging using common hardware and software. The connector can be any connection possible to receptor 300. For example, the connection can be a tether wire going from the ultrasound probe 400 to the receptacle 300, a wireless connection from probe to receptacle, an optical link between the probe and receptacle, or any other means of linking signals between the probe and receptacle. The operator can obtain ultrasound images of particular areas of interest identified by the primary full-filed detector 304.

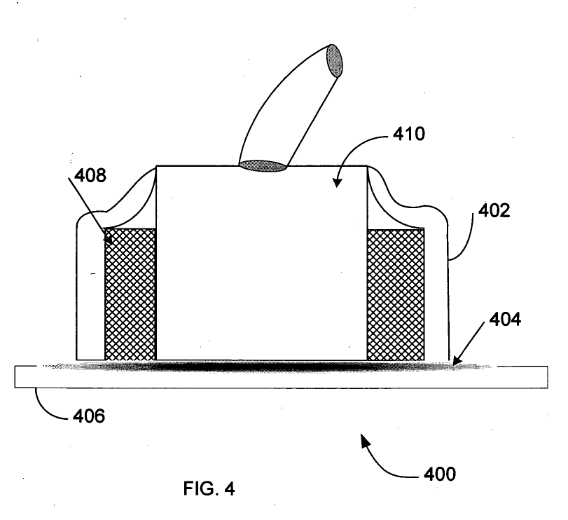

FIG. 4 is a representation of an ultrasound probe 400 that can be connected to the mammography imaging system 100. Ultrasound probe 400 solves the need in the art for complementary imaging using common hardware and software. The ultrasound transducer 400 is surrounded by a skirt or cover 402 that includes a spacer 404 formed along its lower edge. An elastomeric or rubbery material 408, that can facility contact with the ultrasound transducer 400, dampened with a suitable lubricating/coupling fluid, for example, a water-based solution of surfactant and detergent, is disposed around the transducer 410 such that the elastomeric material 408 and the spacer 404 are in contact with compression plate 406 at substantially the same time. Thus, as the transducer assembly moves along the surface of compression plate 406 a thin film of the lubricating/coupling fluid is deposited on the plate of the spacer 404. Cover 402 also permits the transducer assembly to be handled without contacting material 408.

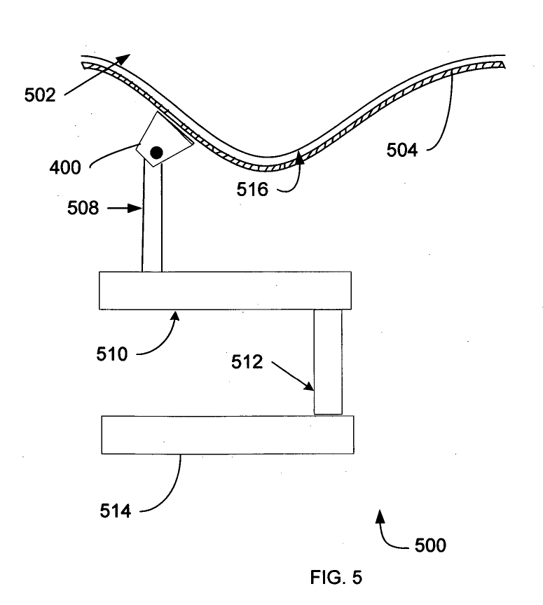

FIG. 5 illustrates the ultrasonic subsystem 500. Imaging system 500 solves the need in the art for generating tomosynthesis images from ultrasound data. The operations of the ultrasonic subsystem 500 uses a partial vacuum to pull the breast into a hollow cavity or chamber, this is to constrain the anatomy in a fixed position without the discomfort of the compression paddle method. The compression is needed for X-ray based imaging because the doctors or operators want the tissue spread as thinly as possible to improve imaging quality. This compression is not needed with ultrasound imaging of the breast. Gel is used within the hollow cavity to eliminate air pockets and to provide a good transmission medium, i.e. acoustic impedance match, at the interface between the cavity and the skin. Such gel may also be needed on the exterior of the cavity shell. The four degrees of freedom: if one imagines an axis coming out of the chest wall, say through the nipple, there's rotation around that axis, distance along that axis, distance from that axis, radial distance perpendicular to the axis, and the fourth is the angle that the ultrasound probe makes to maintain contact approximately perpendicular to the surface of the cavity shell exterior. So that's two linear motions (axial and radial) and two angular motions (one azimuthal of the whole mechanism a full 360 degrees and one angling just the probe, only needs somewhere between 90 to 180 degrees total motion). The idea is to basically provide a motion-control gantry to sweep the probe over the shell in such a way as to get a sufficient data set to provide the desired image.

The subsystem includes an ultrasound probe 400, a motion mechanism 508-514, and chamber 504 for holding a part of a patient's anatomy 502 such as a breast. The purpose of the chamber 504 is to constraint the breast 502 by using a partial vacuum to ensure complete contact of the breast 502 with the chamber 504 surface. A selection of alternative chambers 504 or a chamber 504 with adjustable geometry would be used to provide a close match to individual patient's anatomy 502. If means other than the chamber 504 are used to constraint the patient's anatomy 502 the position of the ultrasound probe 400 could be accomplished by other methods, including manually, if sufficiently accurate data were available about the location (x, y, z coordinates in space) and orientation (angles of the beam relative to the spatial coordinate frame of reference) of the ultrasound probe at all times during the image acquisition.

The motion mechanism has subassembly 508 for moving the ultrasound probe 400 radially along the contour of chamber 504. Additionally, subassembly 510 moves the ultrasound probe 400 axially or inwardly in the direction of the chamber. The full rotation (360 degrees) of the ultrasound probe 400 is accomplished by subassemblies 512 and 514. The four degrees of freedom, respectively, would be: one azimuthal, for the 360 degrees of rotation of the probe around the breast for each tomography slice or set of slices; one linear, along the rotation axis; one radial from the center of rotation, to keep the ultrasound probe in contact with the exterior of the chamber; and one angular, relating probe angle to the rotational axis of the mechanism. Since the ultrasound probe 400 is following the contour of the chamber 504 that substantially is the shape of the anatomy 502 the position of the probe is known for each tomography slice. In the event that other means are used to constraint the anatomy or breast 502 then the position and orientation of the ultrasound probe 400 can be determined by technique described in FIG. 8.

In order to eliminate air pockets between the patient's anatomy 502 and the chamber an ultrasound gel is applied at 506. Ultrasound gel would also be used on the exterior of the chamber 504, and the material of the chamber wall would be selected for appropriate acoustic properties, to minimize attenuation, reflection, or scattering of the beam as it transits the material and interface surfaces. Since the present ultrasound probes 400 are capable of wide fan beam acquisition, data for many computer tomography slices could be acquired in parallel, resulting in only a few axial positions being needed.

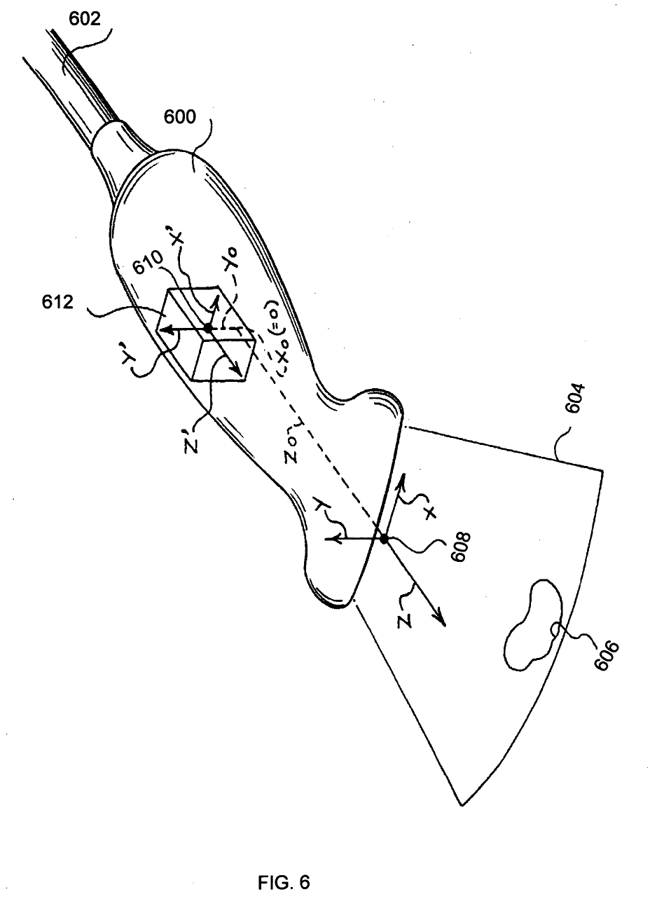

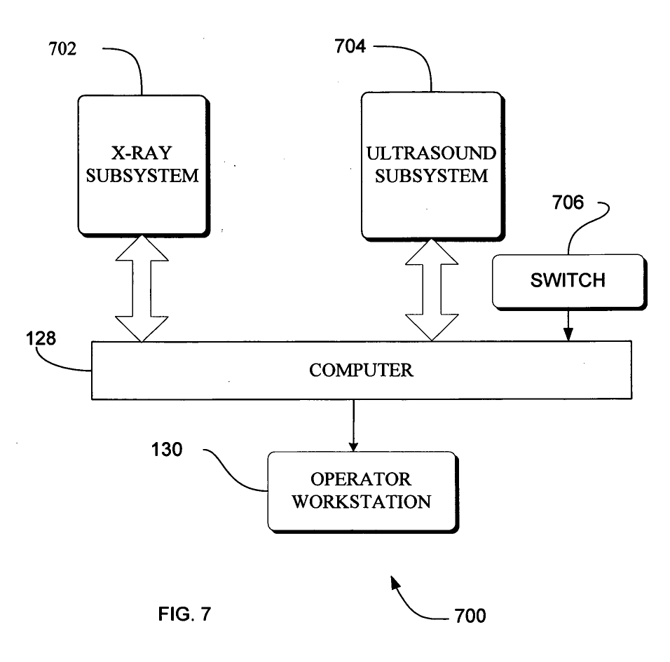

FIG. 6 is an illustration of an ultrasonic transducer probe 600. At least one transducer element (not shown) of the ultrasonic transducer probe 600 generates an image plane 604 for scanning a region of interest 606. Ultrasonic probe 600 satisfies the need in the art for generating tomosynthesis images from ultrasound data. The ultrasonic transducer probe 600 has a position and orientation sensor 612 attached to the housing of the probe 600 to determine the position and orientation of the image plane 604. The sensor can be solid state gyros, piezogyros, or any other know or future discovered device that can directly or indirectly measure location and/or orientation data. Examples of solid state gyros are the Futaba GY240®, the Futaba GY401®, the Futaba GY502® manufactured by the Futaba Corporation. A medical diagnostic ultrasound imaging subsystem (see FIG. 7) coupled with the probe 600 via the probe cable 602 can use the data generated by the sensor 612 to determine the position and orientation of the sensor 612 and/or the image plane 604.

The position and orientation sensor 612 is a either magnetic or optical sensing based on passive or active device attached to or embedded in the device 600 being manipulated, and a set of sensors (not shown), antennae or optical sensors, to determine the location of the device in space relative to the frame of reference of the sensors. The frame of reference for orientation could be a suitable receptacle on the positioner of the breast that would act as a beacon for the ultrasound probe and a holder upon completion of an examination. In general, the sensor probe (612) that monitors the movement of the transducer probe 600 in six degrees of freedom with respect to a transmitter. As shown in FIG. 6, the position and orientation sensor 612 and the transmitter (not shown) in the ultrasonic probe 600 each define an origin (608, 610) defined by three orthogonal axes (X′, Y′, Z′ and X″, Y″, Z″). The sensor 612 monitors the translation of the origin 610 with respect to the origin of the transmitter to determine position and monitors the rotation of the X′, Y′, Z′ axes with respect to the X″, Y″, Z″ axes of the transmitter to determine orientation. The position and orientation of the sensor 612 can be used to determine the position and orientation of the image plane 604. As shown in FIG. 6, the image plane 604 defines an origin 610 defined by three orthogonal axes X, Y, Z, which are preferably aligned with the origin of a center acoustic line generated by the transducer probe 600. The position of the origin 608 and the orientation of axes X′, Y′, Z′ of the position and orientation sensor 612 may not precisely coincide with the position of the origin 608 and the orientation of the axes X, Y, Z of the image plane 604. For example, in FIG. 6, the origin 608 of the image plane 604 is offset from the origin 610 of the position and orientation sensor 612 by a distance Z.sub.0 along the Z-direction and a distance of Y.sub.0 along the Y-direction. Accordingly, the position and orientation of the sensor 612 does not directly describe the position and orientation of the image plane 604.

To determine the position and orientation of the image plane 604 from the position and orientation of the sensor 612, position and orientation sensor calibration data is used to transform the position and orientation of the sensor 612 to the position and orientation of the image plane 604. Accordingly, if the sensor has the same orientation as the image plane, the position and orientation calibration data may not contain any orientation calibration data. Similarly, as shown in FIG. 6, a sensor may not have a positional offset with respect to one or more axes of the image plane. There are a number of ways of defining the image plane/sensor offset, but would require periodic nulling or calibration to a known orientation reference. One method of calibrating at least some types of sensors uses three orthogonal linear dimension offsets in X, Y, Z and three rotation angles about each these axes. Other methods include using a position transformation matrix or quaternions.

The ultrasonic probe 600 for optimal operations requires that the part of the anatomy remains fixed in order to determine the location and orientation of the probe relative to the imaging area. When performing mammography or imaging of the breast, the chamber 504 described in FIG. 5 keeps that part of the anatomy at a fixed location and orientation. The probe 600 and the chamber 504 in combination create an optimal condition for tomographic image reconstruction of the breast. The ultrasound probe 600 requires that the anatomy be held still long enough to get data from enough angles to enable the slice image computations. If the part of the anatomy is held relatively still during data acquisition, such as with breath-hold imaging, the spatial alignment will be sufficient without performing any alignment correction. The correction or spatial alignment processing, as is known to those of ordinary skill in the image rendering art, can be implemented by adding the appropriate functions to the imaging system. However, such correction still requires that the anatomy be held still as much as possible by the patient or by application of a mechanical restrains. For example legs and arms can be secured by mechanical means, abdomen can be secure by the patient holding breath for a period within the imaging cycle, and the neck can be restraint by well known mechanical means in the art.

FIG. 7 illustrates a schematic of the multi modality imaging system 700. The system 700 includes an X-ray mammography imaging subsystem 702 and an ultrasound mammography imaging subsystem 704. Imaging system 700 satisfies the need in the art for complimentary imaging that uses common hardware and software and the need in the art for tomosynthesis images from ultrasound data. These systems may optionally be directly electrically connected to share information, as indicated by the dashed line. The system 700 also contains an image fusion and visualization workstation 130. This workstation 130 may comprise a general or special purpose computer or any other type of image processor. The workstation 130 receives data acquired by the subsystems 702 and 704 through computer 130 to form the image. Preferably, the workstation 130 contains a processor which registers an X-ray image with an ultrasound image and a display with displays a fused X-ray and an ultrasound image.

The X-ray mammography imaging subsystem 702 may comprise any X-ray imaging system, including a 2D X-ray mammography system which uses a digital detector, a 3D X-ray tomosynthesis system, in which the X-ray tube is scanned and a plurality of projection radiographs are acquired from different angles with respect to a stationary breast, or a 3D X-ray CT system in which the X-ray tube is angularly scanned 360 degrees. Likewise, the ultrasound mammography imaging subsystem 704 may comprise any ultrasound imaging system existing or any later developed ultrasound imaging system. Any combination of the above subsystems may comprise the multi modality system 1, including 3D X-ray with 3D ultrasound imaging, 3D X-ray with 2D ultrasound imaging, 2D X-ray with 3D ultrasound imaging, and 2D X-ray with 2D ultrasound imaging.

FIG. 7. illustrates a dual-modality full-featured mammography imaging system 700. The system uses a switch 707 at the mammography system 700 console to select between the X-ray mammography subsystem 702 and the ultrasound subsystem 704. The switch 707 can be conventional switch at the console, a switch at the display of the mammography system, or a software switch that can be selected by use of a keyboard, mouse, touch screen, or automatically selected based on selected conditions. This arrangement would use the high-quality display of the existing mammography system 700 to display ultrasound images when the system was being used in ultrasound mode. The ultrasound console controls would be integrated into the mammography console to make a single unified console. The ultrasound probe would connect to the system with a cable that plugs into the mammography gantry. This provides the need in the art for a simpler and more compact packaging for the user versus two separate systems, making it easier to fit an integrated dual-modality full-featured mammography imaging system into a given user procedure room.

FIG. 8 is a block diagram of mammography imaging system 800. Imaging system 800 satisfies the need in the art for complimentary imaging that uses common hardware and software and the need in the art for tomosynthesis images from ultrasound data. The mammography system 800 includes an X-ray subsystem for performing X-ray imaging, a computer 128 for controlling and performing imaging acquisition for both X-ray or ultrasound images, and workstation 130 for storing, displaying, and image analysis. Item 802 is an ultrasound probe as described more fully with FIG. 6 having a position sensor 806. Ultrasound probe 802 and sensor 806 can be encased together to form the ultrasound subsystem 808 for ultrasound imaging and for ascertaining position data based on the movement of the ultrasound probe for each image taken of the patient's anatomy. A motion controller 804 is shown for positioning the ultrasound probe at a desired location.

Motion controller 804 can be a suitably programmed microprocessor that in combination with position sensor 806 can placed the ultrasound probe in a desired location to perform a tomography slice or set of slices. The motion controller 804 can in combination with an operator position the ultrasound probe 802 at a desired location for imaging.

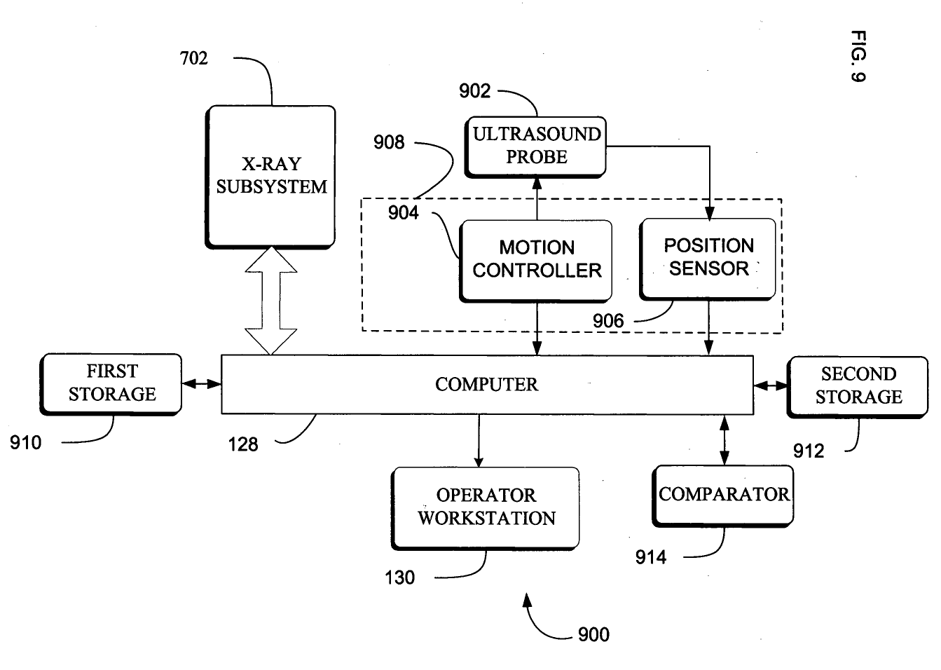

FIG. 9 is a block diagram for a mammography imaging system 900. Imaging system 900 satisfies the need in the art for complimentary imaging that uses common hardware and software and the need in the art for tomosynthesis images from ultrasound data. The imaging system includes an X-ray subsystem 502 and ultrasound subsystem (902, 908) as described in earlier figures. The ultrasound subsystem can be manipulated and placed into position by a combination of machine and human intervention. Thus reference to motion controller 704 is a motor controller or human operator positioning the probe over a desired region.

Mammography imaging system 900 includes a first storage 910, second storage 712, and comparator 714 units for tracking a schedule of images needed for a particular analysis. The analysis could be for the purposes of reconstruction, tomosynthesis, fusion of images, or any other technique that requires a set of images regardless of the modality employed. The first storage 910 has a schedule of images needed for a session by the operator. The session can be based on position and orientation data. For example, a session can be that images from a given location and orientation are desired for a particular analysis or diagnoses. The session, should be understood, can be completed at any point in time or can be delayed until other tests are performed. The second storage 912 would be a collection of images for a given session that have at a minimum an indicia indication location and orientation. For example, an image would indicate the parameters that define the location of imaging space and the orientation of the ultrasound probe 902 relative to the imaging space. Of with probe locations and orientations known for a set of image data taken over a sufficient set of orientations, tomography image reconstructions can be computed to provide tomography images and/or 3-D images from this data set. In this arrangement, the operator manipulating the ultrasound probe effectively substitutes for the CT gantry, moving the probe in a manner so as to obtain a sufficient set of data to perform the image reconstructions to the desired level of image quality. A comparator 914 using the schedule data in the first storage 710 and the imaged information in the second storage 912 can track the locations and orientations already covered by the probe. The comparator 914 can be physical circuit or it can be software that could cue the operator as to what locations and orientations of the probe remain needed to provide sufficient data to complete the image reconstructions, thus guiding the operator's manipulations of the probe. In this way the manual skill of the human operator, who is good at maintaining the contact of the probe to the patient without excess pressure or discomfort to the patient, can be combined with the thoroughness of a computer, to enable sufficient data acquisition as required by the computer to successfully complete tomography reconstruction and/or 3-D image synthesis from the data.

### Methods of an Embodiment

In the previous section, a system level overview of the operation of an embodiment was described. In this section, the particular methods performed by the server and the clients 128 and 130 of such an embodiment are described by reference to a series of flowcharts. Describing the methods by reference to a flowchart enables one skilled in the art to develop such programs, firmware, or hardware, including such instructions to carry out the methods on suitable computerized clients the processor of the clients executing the instructions from computer-readable media. Similarly, the methods performed by the server computer programs, firmware, or hardware are also composed of computer-executable instructions. Methods 1100-150000 are performed by a client program executing on, or performed by firmware or hardware that is a part of a computer, a microprocessor, or controller and is inclusive of the acts required to be taken by the computer 128 or workstation 130.

FIG. 11 is a flowchart of a method 1100 performed by a computer 128 or a workstation 130 according to an embodiment. Method 1100 satisfies the need in the art for examining a selected area without biopsy. Method 1100 controls the mammography system enumerated in the prior figures to acquire X-ray data by use of different detectors.

The method begins with action 1102. In action 1102 the mammography system is commanded to irradiate a breast with X-rays for a certain period of time. Additionally, action 1102 read the output of the detector in receptacle 112 so as to form an image of the breast. In addition to reading the impinging X-rays on the detector, action acquires additional information such as region of interest, position of the detector within the receptacle, and the depth of tissue that may require further analysis. The position of the detector is known as road map data and the purpose is to define the location of a first detector within the receptacle as described by different degrees of freedom. The degree of freedom can be left or right from a given marking, up or down from a given marking, or outward or inward from a defined level. More formally an arbitrary space within the receptacle can be defined by Cartesian coordinates such as X, Y, Z, which leads to six (6) degrees of freedom. Further, an arrangement with fewer degrees of freedom, for example 2, can still be used to position a second sensor. Control passes to action 1104.

Action 1104 acquires a first dataset. The first dataset contains signals such as intensity of X-rays, depth signals, and roadmap signals. Control passes to action 1106 for further processing.

In action 1106 information is derived. The derived information concerns depth of tissue, roadmap or the location to position a second detector for a higher DQE image, and conversion of intensity to an image viewable on a display with adequate resolution. Control then passes to action 1108.

In action 1108 irradiation and detection is undertaken. In actions 1104 and 1106 or by a user, for example a doctor or mammography technician, a region was identified for further analyses with a more superior image then the one derived from the first detector. Using the road map data the computer or the operator can position the second detector for taking the second image. The X-ray source is use to irradiate the breast and the second detector measures the intensity of the transmitted X-rays. Control then passes to action 1110.

In action 1110 the second dataset is acquired. The acquired dataset is processed by the computer 128 or workstation 130 an image of the irradiated region is produced. Control then passes to action 1112 for further processing.

In action 1112 the datasets are visualize on a high resolution display. The images can be viewed individually or combined together into a single display. In the alternative, a workstation with dual monitor could be used to view the images in different screens.

FIG. 12 is a flowchart of a method 1200 performed by a computer 128 or a workstation 130 according to an embodiment. Method 1200 satisfies the need in the art for complementary imaging having common hardware and software. The purpose of the method is to use as much image detection and display electronics with dual modality capabilities. Instead of using discrete units for ultrasound and X-ray, the method uses the components of the X-ray system to process and display ultrasound images.

The method begins with action 1202 with selection of modality. As noted earlier with reference to switch 706, the modality may be selected by a software trigger or by the activation of a physical switch at the console of the mammography system 700. The software trigger could be based on statistical analysis based prior uses, activation switch at the ultrasound probe, or a myriad of other possibilities. After the modality has been selected control passes to action 1204.

In action 1204 the ultrasound modality is determined. Action 1204 decides whether or not the ultrasound modality was selected in action 1202. It should be understood that action 1204 could have as easily tried to determine if an X-ray modality was selected. If an ultrasound modality was selected then control passes to action 1206 or control passes to action 1208.

In action 1206 ultrasound data is acquired. The ultrasound data can be acquired by following methods 1300, 1400, or 1500. If the modality selected had been X-ray then the data would be acquired by the known methods for acquiring X-ray data or by method 1100. Once the data is acquired, X-ray or ultrasound data, control passes to action 1210.

In action 1210 an image is created. The created image can be an X-ray image or ultrasound image. Further, note that action 1210 realizes that notwithstanding the modality the rest of the electronics in the imaging receptor and imaging acquisition electronics (ref-reg board, detector control board, and imaging detector circuit (IDC)) can be used commonly by both modalities. Control then passes to action 1212.

In action 1212 the created image is stored. The image can be preserved in long term and short term storage. The conventional size for an image is 8 MB and normally there are eight images per session (64 MB) so short term memory could be RAM, ZIP drive, or hard drive at the computer 128 or workstation 130. Long term storage could be accomplished through picture archiving and communication system (PACS) that is well known to those in the art. After the image is stored control passes to action 1214 for further processing.

In action 1214 the image is displayed. The images should be displayed with a grey scale that is near optimal requiring minimal manipulation. Different workstations have different capacities in this respect. The General Electric review workstation can display 8 bits, which means 256 levels of grey. The eye can perceive only about 150 levels of grey. The problem is then not the number of grey levels presented, but to see that they contain the information that is needed for the imaging task. If a 14 bit digital image is compressed to a 10-bit representation, only 1/16 of the full grey scale can be seen in one presentation with full grey scale resolution. With an 8-bit representation, only 1/64 of the full grey scale can be seen correspondingly. It is therefore necessary to extract the information to be presented very carefully. One possible solution as for the General electric review workstation is the use of several different window levels that can be quickly selected on a special keyboard.

FIG. 13 is a flowchart of a method 1300 performed by a computer 128 or a workstation 130 according to an embodiment. Method 300 satisfies the need in the art for tomosynthesis images from ultrasound data. The objective of the method is to acquire ultrasound image data of the anatomy from a full revolution (360 degrees) of beam perspective.

The method begins with action 1302 of positioning the anatomy in the chamber. As noted earlier with reference to FIG. 5 the breast is held in place by a chamber that can be adjusted or designed to the shape of the subject by the use of a vacuum. Further, in order to enhance the quality of the image a gel can be applied in the inner and outer portion of the chamber so as to eliminate air gaps that can reduce the overall quality of the ultrasound image through attenuation, reflection, or scattering of the ultrasound beam. After the breast has been position in the chamber control then passes to action 1304.

In action 1304 the contour of the chamber is scan by the use of an ultrasound probe. A moving mechanism that can be servo or manually controlled follows the contour of the chamber. At a minimum the movement should follow four degrees of freedom based on azimuthal for the 360 degrees of rotation for each set of slices, linear along the rotational axis, radial from the center of rotation, and angular relating probe angle to the rotational axis of the moving mechanism. After the mechanism has performed its gyrations around the chamber the acquired data is assembled into ultrasound data ready to be converted to an image in action 1306.

In action 1308 and image is created. In action 1308 the data points acquired are converted to an image. Control then passes to action 1310.

In action 1310 a determination is made as to completion of imaging for the particular session. If imaging is not completed then control passes to action 1304 for further processing. If imaging is completed then the image or images are stored for further analysis or viewing.

In action 1312 the created image or images are stored. The storage of the images is either in long or short term storage as noted in earlier descriptions of methods 1100 and 1200. After the action of storage is completed control passes to action 1314 for further processing.

In action 1314 the image or images of the breast are display on a suitable display for analysis.

FIG. 14 is a flowchart of a method 1400 performed by a computer 128 or a workstation 130 according to an embodiment. Method 1400 satisfies the need in the art for tomosynthesis images from ultrasound data. The objective of the method is acquire ultrasound image data of the anatomy from a full revolution (360 degrees) of beam perspective by use of an ultrasound probe on a breast that is constraint by means other than chamber 504. The positioning of the ultrasound probe could be accomplished by other methods, including manually, if sufficiently accurate data were available about location (X, Y, Z coordinates) and orientation. An ultrasound probe, see FIG. 6, which can determine its location and orientation would accomplish this necessary condition.

Method 1400 begins with action 1402. In action 402, sensors in probe 600 acquire the location and orientation of the ultrasound probe relative to the breast being inspected. After these signals are acquired control passes to action 1404 for further processing.

In action 1404, the acquired location and orientation signals are corrected. The correction can be performed by either table lookup, mathematical manipulation of the signals, filtering, or any known or future techniques for correcting signals. Further, both the acquiring of the signals and the correcting of the signals can reside in the ultrasound probe 600. In the alternative the correcting can be performed by appropriate circuitry or software in the mammography system. After the signal is corrected control passes to action 1408 for further processing.

In action 1406 the corrected signal is obtained and processed to create an ultrasound image. When the dataset has been acquired control passes to action 1408.

In action 1410 the created image or images are stored. The storage of the images is either in long or short term storage as noted in earlier descriptions of methods 1100 and 1200. After the action of storage is completed control passes to action 1412 for further processing.

In action 1412 a determination is made as to completion of imaging for the particular session. If imaging is not completed then control passes to action 1402 for further processing. If imaging is completed then control passes to action 1414 for further processing.

In action 1414 the image or images of the breast are display on a suitable display for analysis.

FIG. 15 is a flowchart of a method 1500 performed by a computer 128 or a workstation 130 according to an embodiment. Method 1500 satisfies the need in the art for tomosynthesis images from ultrasound data. The objective of the method is acquire image data by following a schedule or maintaining a list of location and orientation perspective in order to form a three dimensional representation of the breast.

The method begins with action 1502. In action 1502 the operator, user, or computer system enters a schedule of images needed to acquire a three dimensional representation of the breast. The schedule as used here can include the sequence by which the images have to be taken or it can additionally be defined based on location and orientation of the probe relative to the breast. Once the schedule has been received control then passes to action 1504.

In action 1504, imaging is conducted by the mammography system following any of the preceding methods such as 1100, 1200, 1300, or 1400. Once the image has been acquired then control passes to action 1506.

In action 1506 and indicia is applied to the image. The indicia can be any label that facilitates comparison with the schedule enumerated in action 1502. For example, the indicia could be based on location and orientation of an ultrasonic probe or the indicia could be an alphanumeric sequence that can be compared against the schedule. After indicia is affixed to the image control passes to action 1508.

In action 1508 a comparison is made of the imaging schedule and the indicia of the images that have been performed. If there is an indication that other images need to be taken then actions 1504, 1506, and 1508 are repeated until all the items in the imaging schedule match the indicia applied to exposed images. The indication can be done by maintaining a buffer, table, or list that is either removed or flagged for completion by the system.

In action 1510 a 3-D representation of the breast is visualize on a suitable display for analysis.

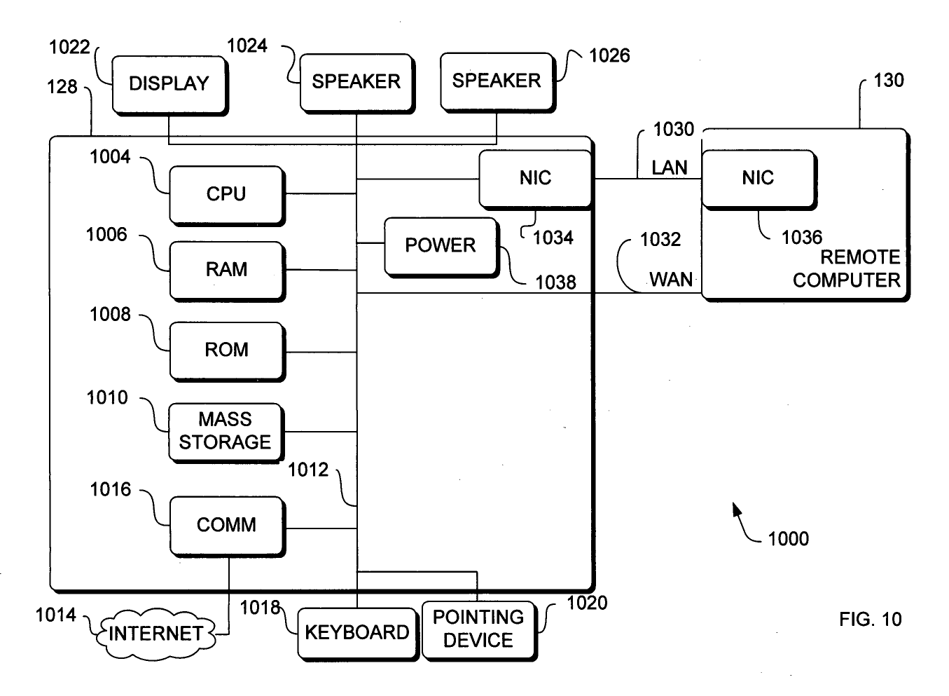

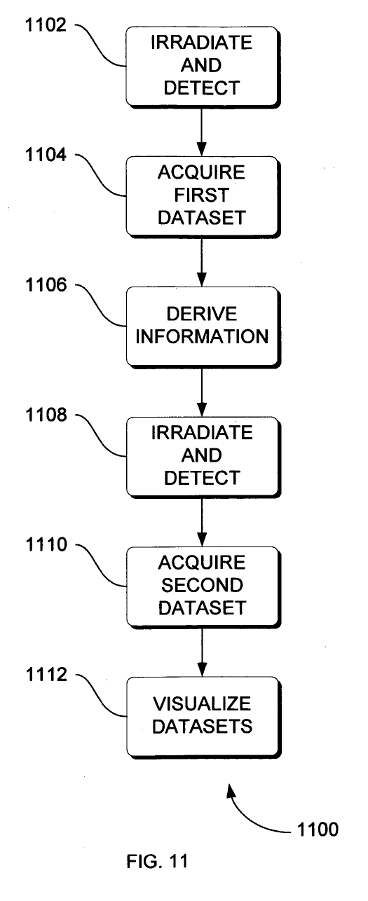

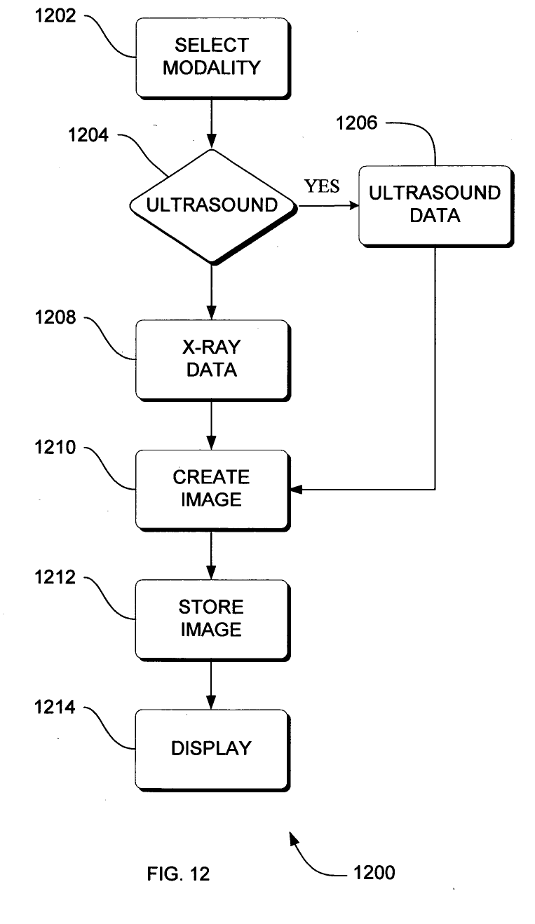

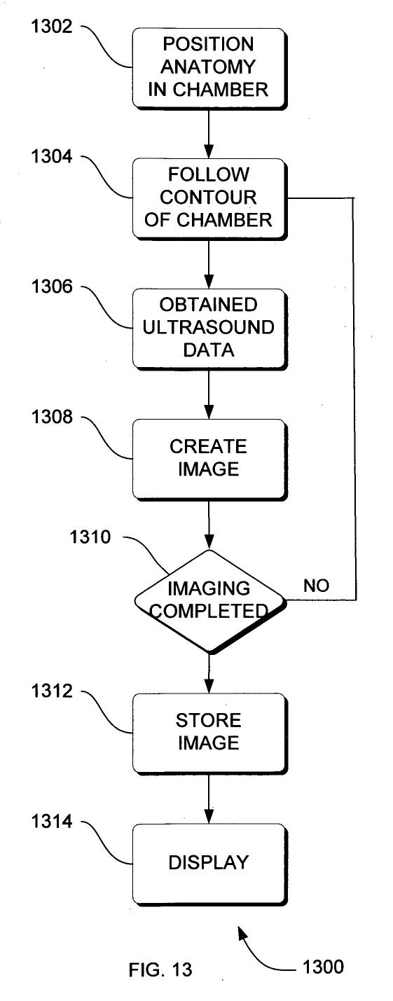

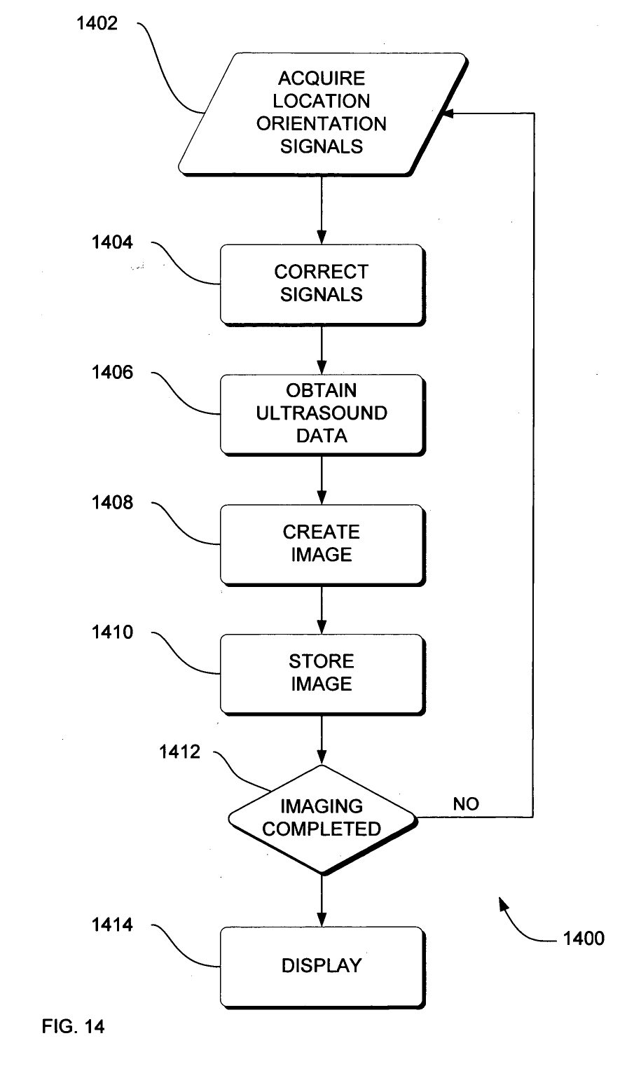

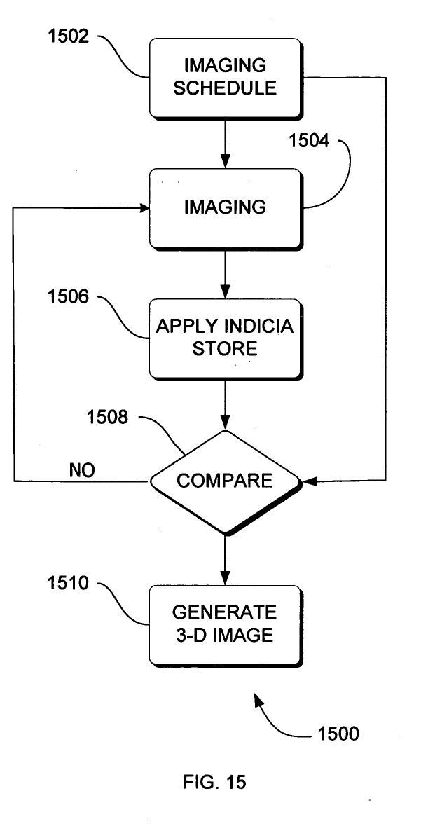

In some embodiments, methods 1100-1500 are implemented as a computer data signal embodied in a carrier wave, that represents a sequence of instructions which, when executed by a processor, such as processor 1004 in FIG. 10, cause the processor to perform the respective method. In other embodiments, methods 1100-1400 are implemented as a computer-accessible medium having executable instructions capable of directing a processor, such as processor 1004 in FIG. 10, to perform the respective method. In varying embodiments, the medium is a magnetic medium, an electronic medium, or an optical medium.

### Hardware and Operating Environment

FIG. 10 is a block diagram of the hardware and operating environment 1000 in which different embodiments can be practiced. The description of FIG. 10 provides an overview of computer hardware and a suitable computing environment in conjunction with which some embodiments can be implemented. Embodiments are described in terms of a computer executing computer-executable instructions. However, some embodiments can be implemented entirely in computer hardware in which the computer-executable instructions are implemented in read-only memory. Some embodiments can also be implemented in client/server computing environments where remote devices that perform tasks are linked through a communications network. Program modules can be located in both local and remote memory storage devices in a distributed computing environment.

Computer 1002 includes a processor 1004, commercially available from Intel, Motorola, Cyrix and others. Computer 1002 also includes random-access memory (RAM) 1006, read-only memory (ROM) 1008, and one or more mass storage devices 1010, and a system bus 10102, that operatively couples various system components to the processing unit 1004. The memory 1006, 1008, and mass storage devices, 1010, are types of computer-accessible media. Mass storage devices 1010 are more specifically types of nonvolatile computer-accessible media and can include one or more hard disk drives, floppy disk drives, optical disk drives, and tape cartridge drives. The processor 1004 executes computer programs stored on the computer-accessible media.

Computer 1002 can be communicatively connected to the Internet 1014 via a communication device 1016. Internet 1014 connectivity is well known within the art. In one embodiment, a communication device 1016 is a modem that responds to communication drivers to connect to the Internet via what is known in the art as a “dial-up connection.” In another embodiment, a communication device 1016 is an Ethernet® or similar hardware network card connected to a local-area network (LAN) that itself is connected to the Internet via what is known in the art as a “direct connection” (e.g., T1 line, etc.).

A user enters commands and information into the computer 1002 through input devices such as a keyboard 10110 or a pointing device 1020. The keyboard 10110 permits entry of textual information into computer 1002, as known within the art, and embodiments are not limited to any particular type of keyboard. Pointing device 1020 permits the control of the screen pointer provided by a graphical user interface (GUI) of operating systems such as versions of Microsoft Windows®. Embodiments are not limited to any particular pointing device 1020. Such pointing devices include mice, touch pads, trackballs, remote controls and point sticks. Other input devices (not shown) can include a microphone, joystick, game pad, satellite dish, scanner, or the like.

In some embodiments, computer 1002 is operatively coupled to a display device 1022. Display device 1022 is connected to the system bus 1012. Display device 1022 permits the display of information, including computer, video and other information, for viewing by a user of the computer. Embodiments are not limited to any particular display device 1022. Such display devices include cathode ray tube (CRT) displays (monitors), as well as flat panel displays such as liquid crystal displays (LCD's). In addition to a monitor, computers typically include other peripheral input/output devices such as printers (not shown). Speakers 1024 and 1026 provide audio output of signals. Speakers 1024 and 1026 are also connected to the system bus 1012.

Computer 1002 also includes an operating system (not shown) that is stored on the computer-accessible media RAM 1006, ROM 1008, and mass storage device 1010, and is and executed by the processor 1004. Examples of operating systems include Microsoft Windows®, Apple MacOS®, Linux®, UNIX®. Examples are not limited to any particular operating system, however, and the construction and use of such operating systems are well known within the art.

Embodiments of computer 1002 are not limited to any type of computer 1002. In varying embodiments, computer 1002 comprises a PC-compatible computer, a MacOS®-compatible computer, a Linux®-compatible computer, or a UNIX®-compatible computer. The construction and operation of such computers are well known within the art.

Computer 1002 can be operated using at least one operating system to provide a graphical user interface (GUI) including a user-controllable pointer. Computer 1002 can have at least one web browser application program executing within at least one operating system, to permit users of computer 1002 to access intranet or Internet world-wide-web pages as addressed by Universal Resource Locator (URL) addresses. Examples of browser application programs include Netscape Navigator® and Microsoft Internet Explorer®.

The computer 128 can operate in a networked environment using logical connections to one or more remote computers, such as remote computer 130. These logical connections are achieved by a communication device coupled to, or a part of, the computer 128. Embodiments are not limited to a particular type of communications device. The remote computer 130 can be another computer, a server, a router, a network PC, a client, a peer device or other common network node. The logical connections depicted in FIG. 10 include a local-area network (LAN) 1030 and a wide-area network (WAN) 1032. Such networking environments are commonplace in offices, enterprise-wide computer networks, intranets and the Internet.

When used in a LAN-networking environment, the computer 128 and remote computer 130 are connected to the local network 1030 through network interfaces or adapters 1034, which is one type of communications device 1016. Remote computer 130 also includes a network device 1036. When used in a conventional WAN-networking environment, the computer 128 and remote computer 130 communicate with a WAN 1032 through modems (not shown). The modem, which can be internal or external, is connected to the system bus 10102. In a networked environment, program modules depicted relative to the computer 1002, or portions thereof, can be stored in the remote computer 130.

Computer 128 also includes power supply 1038. Each power supply can be a battery.

More specifically, in the computer-readable program embodiment, the programs can be structured in an object-orientation using an object-oriented language such as Java, Smalltalk or C++, and the programs can be structured in a procedural-orientation using a procedural language such as COBOL or C. The software components communicate in any of a number of means that are well-known to those skilled in the art, such as application program interfaces (API) or interprocess communication techniques such as remote procedure call (RPC), common object request broker architecture (CORBA), Component Object Model (COM), Distributed Component Object Model (DCOM), Distributed System Object Model (DSOM) and Remote Method Invocation (RMI). The components execute on as few as one computer as in computer 128 in FIG. 10, or on at least as many computers as there are components.

### CONCLUSION

A mammography system and method has been described. Although specific embodiments have been illustrated and described herein, it will be appreciated by those of ordinary skill in the art that any arrangement which is calculated to achieve the same purpose may be substituted for the specific embodiments shown. This application is intended to cover any adaptations or variations.

In particular, one of skill in the art will readily appreciate that the names of the methods and apparatus are not intended to limit embodiments. Furthermore, additional methods and apparatus can be added to the components, functions can be rearranged among the components, and new components to correspond to future enhancements and physical devices used in embodiments can be introduced without departing from the scope of embodiments. One of skill in the art will readily recognize that embodiments are applicable to future communication devices, different file systems, and new data types.

We claim:

1. A mammography system having an X-ray source, a breast compression plate, and a digital image receptor, the receptor comprising:
a movement mechanism;
a first detector coupled to the movement mechanism operable to receive energy from said X-ray source and for providing roadmap data and X-ray source data; and
a second detector coupled to the movement mechanism operable to receive X-ray source energy and for providing X-ray source data.

2. The mammography system of claim 1, wherein the movement mechanism is a three degrees of freedom mechanism.

3. The mammography system of claim 1, wherein the movement mechanism positions the second detector based on the roadmap data.

4. The mammography system of claim 3, wherein the second detector is at least one of a direct conversion device, a charge coupled device, and an optoelectric device.

5. A mammography system comprising:
an X-ray source;
a breast compression plate; and
a digital image receptor, the receptor comprising:
a first detector receiving energy from said X-ray source and for providing X-ray source data; and
an electrical connector capable of coupling at least one external device.

6. The mammography system of claim 5, wherein the external device further comprises:
an ultrasound probe further comprising an ultrasonic transmitter and an ultrasonic detector.

7. The mammography system of claim 5, further comprising:
a gel pad acoustically coupled to the ultrasonic transducer.

8. The mammography system of claim 7, further comprising:
an enclosure that encapsulates the gel pad.

9. The mammography system of claim 8 wherein the gel pad further comprises:
an adherent surface.

10. A mammography system further comprising:
an X-ray source;
a breast compression plate; and
a digital image receptor, comprising:
a first detector receiving energy from said X-ray source and operable to provide X-ray source data;
at least one ultrasonic detector externally coupled to the digital image receptor; and
a ultrasonic transmitter externally coupled to the digital image receptor wherein ultrasonic measurements from the ultrasonic transmitter and ultrasonic detector are used in constructing an image of a patient's breast by the mammography system.

11. The mammography system of claim 10, further comprising:
a gel pad acoustically coupled to the ultrasonic transducer.

12. The mammography system of claim 11, further comprising:
an enclosure that encapsulates the gel pad.

13. The mammography system of claim 10 wherein the gel pad includes an adherent surface.

14. A mammography imaging system, comprising:
an X-ray mammography imaging subsystem adapted to image a breast;
an ultrasound mammography imaging subsystem adapted to image a breast;
a selector switch for selecting between the X-ray mammography imaging subsystem and the ultrasound mammography imaging subsystem;
a device configured to obtain and store data from the selected imaging subsystem; and
a display device operable to display at least one image obtained or stored by said device.

15. The mammography system of claim 14, the system further comprising:
a gantry comprising at least one connector for coupling the ultrasound mammography subsystem to the device configured to obtain and store data.

16. The mammography system of claim 14, wherein the selector switch is one of a toggle switch, a rocker switch, a push button switch, and a lever.

17. The mammography system of claim 14, wherein the device configured to obtain and store data is at least one of a computer, workstation, a microprocessor, a personal digital assistance, and a server.

18. An apparatus for generating a three-dimensional ultrasound image, the apparatus comprising:
an ultrasound probe for generating ultrasound image data of a part of an anatomy through spatial registration with the part of an anatomy;
a motion control system for movement of the probe in relation to the part of an anatomy and for sensing the probe's position, the motion control system including a first-axis control, a second-axis control, a third-axis control, and a fourth axis control for movement of the probe; and
a computer for generating the three-dimensional ultrasound image from the ultrasound image data and from information regarding the spatial registration.

19. An apparatus for generating a three-dimensional ultrasound image of a part of an anatomy, the apparatus comprising:
a first storage device for storing an imaging schedule, the imaging schedule defined by location and orientation;
an ultrasound probe for generating ultrasound image data of the part of an anatomy with indicia indicating location and orientation relative to a part of an anatomy;
a motion control system for movement of the ultrasound probe in relation to the part of an anatomy and for sensing the probe's position, the motion control system including a first-axis control, a second-axis control, a third-axis control, and a fourth axis control for movement of the probe;
a second storage device for storing location and orientation of imaged data;
a comparator for comparing imaged data and imaging schedule and generating and indication of completion or at least one location and orientation; and
a computer for generating the three-dimensional ultrasound image from the ultrasound image data upon the indication of completion or at least one location and orientation.

20. An apparatus for generating ultrasound image of a breast, the apparatus comprising:
a hollow cavity for holding a breast in place so as to be imaged by an ultrasound probe;
a motion system for moving an ultrasound probe in relation to the breast in the hollow cavity;
a ultrasound probe for generating ultrasound image data of the breast in the hollow cavity; and
a computer for generating an ultrasound image from the ultrasound image data and from information regarding the spatial registration.

21. The apparatus of claim 20, wherein the hollow cavity holds the breast in place by applying a partial vacuum between the inner surface of the hollow cavity and the breast to be imaged by the ultrasound probe.

22. The apparatus of claim 21, wherein the motion system is a four degrees of freedom mechanism.

23. The apparatus of claim 22, wherein the degrees of freedom are azimuthal, linear, radial, and angular.

24. An ultrasound system further comprising an ultrasound probe, the ultrasound probe comprising:
a sensor capable of providing signals that represent position and orientation; and
a device capable of correcting the position and orientation signals and capable of generating signals that represent the actual position and orientation of the ultrasound probe relative to an object.

25. A mammography method perform on a mammography system further comprising a receptor with dual X-ray detectors, comprising:
irradiating a breast with X-rays and detect the X-rays transmitted through the breast with a first detector;
acquiring at least one first data set of X-ray from the first detector and form a first image of the X-ray from the data set;
deriving information from the first data set to acquire a second data set;
irradiating a breast with X-rays and detect the X-rays transmitted through the breast with a second detector;
acquiring at least one second data set of X-ray from the second detector and form a second image of the X-ray from the data set; and
visualizing at least one of first image and second image on an information medium.

26. The method of claim 25, wherein the information from the first data set includes one of road map data, depth data, region of interest data.

27. The method of claim 25, wherein the first detector and the second detector share a receptacle.

28. The method of claim 27, wherein visualizing is one of displaying first and second image, combining first and second image, fusing first and second image.

29. A mammography method performed by a mammography system further comprising a receptacle with an X-ray detector and connector for an ultrasonic probe, the mammography method comprising:
irradiating a breast with X-rays and detecting the X-rays transmitted through the breast with the X-ray detector;
acquiring at least one first data set of X-ray from the first detector and forming a first image of the X-ray from the data set;
coupling an ultrasound probe to the connector in the receptor of the mammography system;
applying ultrasound energy to the breast and detecting reflected ultrasound energy;
acquiring at least one second data set of ultrasound energy from the ultrasound probe and forming a second image from the data set; and
visualizing at least one of first image and second image on an information medium.

30. The method of claim 29, further comprising:
fusing the first image and the second image to form a composite three dimensional image; and
displaying the fused image.

31. The method of claim 30, wherein fusion of the first and the second image is based on mechanically co-registered acquisition, co-registered acquisition supplemented by imaging physics or mutual information based registration.

32. The method of claim 29, further comprising:
using information from the first data set to acquire the second data set.

33. The method of claim 29, further comprising:
using information from the first data set to optimize quality of the second image.

34. A mammography imaging method performed by a mammography system further comprising a X-ray imaging subsystem and ultrasound imaging subsystem, the mammography imaging method comprising:
selecting between the X-ray mammography imaging subsystem and ultrasound mammography imaging subsystem to image a breast;
obtaining data from the selected imaging subsystem;
creating an image representation of the obtained data from the selected imaging subsystem;
storing the image representation from the selected imaging subsystem; and
displaying created or stored image representation.

35. The method of claim 34, the method further comprising:
electrically coupling the ultrasound mammography subsystem at a gantry mechanism located in the X-ray subsystem.

36. The method of claim 34, wherein the action of selecting is accomplished through one of a toggle switch, rocker switch, push button switch, and lever.

37. The method of claim 34, wherein the actions of selecting, obtaining, creating, storing are accomplished by least one of computer, workstation, microprocessor, personal digital assistance, and server.

38. The method of claim 34, wherein the action of storing is one or more images from each selected imaging subsystem to form a first and second image.

39. The method of claim 38, further comprising:
fusing the first image and the second image to form a composite three dimensional image; and
displaying the fused image.

40. The method of claim 39, wherein fusion of the first and the second image is based on mechanically co-registered acquisition, co-registered acquisition supplemented by imaging physics or mutual information based registration.

41. The method of claim 38, further comprising:
using information from the X-ray subsystem to acquire at least one image from the ultrasound subsystem.

42. The method of claim 38, further comprising:
using information from the X-ray subsystem to optimize the quality of the second image from the ultrasound subsystem.

43. A mammography method performed by a mammography system further comprising a breast shaped chamber for constraining a breast, the mammography method comprising:
positioning the breast to be imaged in the chamber;
moving an ultrasound probe outside the breast shaped chamber to a desired location so as to image the breast;
applying ultrasound energy to the breast and detecting reflected ultrasound energy;
obtaining data from the reflected ultrasound energy;
creating an image representation of the obtained data from the reflected ultrasound energy;
storing the image representation from the reflected ultrasound energy; and
displaying created or stored image representation.

44. The method of claim 43, wherein the action of storing is one or more images from the reflected ultrasound energy.

45. The method of claim 44, further comprising:
fusing the first image and the second image to form a composite three dimensional image; and
displaying the fused image.

46. The method of claim 44, wherein fusion of the first and the second image is based on mechanically co-registered acquisition, co-registered acquisition supplemented by imaging physics or mutual information based registration.

47. The method of claim 44, further comprising:
using information from a previous image to acquire a subsequent image.

48. The method of claim 44, further comprising:
using information from at least one previous image to optimize the quality of subsequent images.

49. A method performed by a medical imaging system, the method comprising:
sensing location and orientation signals of an ultrasound probe relative to a part of an anatomy to be imaged;
correcting the sensed location and orientation signals of the ultrasound probe relative to a part of an anatomy to be imaged;
applying ultrasound energy to the part of an anatomy and detecting reflected ultrasound energy;
obtaining data from the reflected ultrasound energy and corrected sensed location and orientation signals;
creating an image representation of the obtained data from the reflected ultrasound energy;
storing the image representation from the reflected ultrasound energy; and
displaying created or stored image representation.

50. The method of claim 49, wherein the action of storing is one or more images from the reflected ultrasound energy.

51. The method of claim 50, further comprising:
fusing the first image and the second image to form a composite three dimensional image; and displaying the fused image.

52. The method of claim 50, wherein fusion of the first and the second image is based on mechanically co-registered acquisition, co-registered acquisition supplemented by imaging physics or mutual information based registration.

53. The method of claim 50, further comprising:
using information from a previous image to acquire a subsequent image.

54. The method of claim 50, further comprising:
using information from at least one previous image to optimize the quality of subsequent images.

55. A medical imaging method performed by a medical imaging system further comprising an ultrasound probe, the method comprising:
applying ultrasound energy to a part of an anatomy and detecting reflected ultrasound energy;
receiving information from the ultrasound probe indicative of location and orientation relative to the part of an anatomy;
obtaining data from the reflected ultrasound energy and received information indicative location and orientation signals;
creating an image representation of the obtained data from the reflected ultrasound energy;
storing the image representation from the reflected ultrasound energy; and
displaying created or stored image representation.

56. The method of claim 55, wherein the action of storing is one or more images from the reflected ultrasound energy.

57. The method of claim 56, further comprising:
fusing the first image and the second image to form a composite three dimensional image; and
displaying the fused image.

58. The method of claim 56, wherein fusion of the first and the second image is based on mechanically co-registered acquisition, co-registered acquisition supplemented by imaging physics or mutual information based registration.

59. The method of claim 56, further comprising:
using information from a previous image to acquire a subsequent image.

60. The method of claim 56, further comprising:
using information from at least one previous image to optimize the quality of subsequent images.

61. The method of claim 56, wherein the received information comprises,
sensing location and orientation signals of an ultrasound probe relative to a part of an anatomy to be imaged; and
correcting the sensed location and orientation signals of the ultrasound probe relative to a part of an anatomy to be imaged.

62. A method for generating a three-dimensional ultrasound image of a part of an anatomy, the method comprising:
storing an imaging schedule defined by location and orientation of an ultrasound probe;
moving the ultrasound probe to a position that is defined by a location and an orientation;
generating at least one ultrasound image with an indicia indicating location and orientation;
storing the indicia that are indicative of location and orientation of the ultrasound image;
storing the generate ultrasound image with an indicia indicating location and orientation;
comparing the stored indicia and the stored imaging schedule;
generating an indication of completion based on the comparison of the stored indicia and the stored imaging schedule;
repeating the previous actions if the indication is non completion; and
generating a three-dimensional ultrasound image from the store ultrasound image upon the indication of completion.

63. A computer-accessible medium having executable instructions to control the operations of a medical imaging system, the executable instructions capable of directing a processor to perform:
storing an imaging schedule defined by location and orientation of an ultrasound probe;
moving the ultrasound probe to a position that is defined by a location and an orientation;
generating at least one ultrasound image with an indicia indicating location and orientation;
storing the indicia that are indicative of location and orientation of the ultrasound image;
storing the generate ultrasound image with an indicia indicating location and orientation;
comparing the stored indicia and the stored imaging schedule;
generating an indication of completion based on the comparison of the stored indicia and the stored imaging schedule;
repeating the previous actions if the indication is non completion; and
generating a three-dimensional ultrasound image from the store ultrasound image upon the indication of completion.

64. A computer-accessible medium having executable instructions to control the operations of a medical imaging system, the executable instructions capable of directing a processor to perform
sensing location and orientation signals of an ultrasound probe relative to a part of an anatomy to be imaged;
correcting the sensed location and orientation signals of the ultrasound probe relative to a part of an anatomy to be imaged;
applying ultrasound energy to the breast and detecting reflected ultrasound energy;
obtaining data from the reflected ultrasound energy and corrected sensed location and orientation signals;
creating an image representation of the obtained data from the reflected ultrasound energy; and
storing the image representation from the reflected ultrasound energy; and
displaying created or stored image representation.

65. The method of claim 64, wherein the action of storing is one or more images from the reflected ultrasound energy.

66. The method of claim 65, further comprising:
fusing the first image and the second image to form a composite three dimensional image; and
displaying the fused image.

67. The method of claim 65, wherein fusion of the first and the second image is based on mechanically co-registered acquisition, co-registered acquisition supplemented by imaging physics or mutual information based registration.

68. The method of claim 65, further comprising:
using information from a previous image to acquire a subsequent image.

69. The method of claim 65, further comprising:
using information from at least one previous image to optimize the quality of subsequent images.

70. A computer data signal embodied in a digital data stream comprising data including a representation of a first image, the first image comprising a first plurality of pixels, wherein the computer data signal is generated by a method comprising:
applying ultrasound energy to a part of an anatomy and detecting reflected ultrasound energy;
receiving information from the ultrasound probe indicative of location and orientation relative to the part of an anatomy;
obtaining data from the reflected ultrasound energy and received information indicative location and orientation signals; and
creating an image representation of the obtained data from the reflected ultrasound energy.

71. The method of claim 70, further comprising:
fusing a first image and a second image to form a composite three dimensional image.

72. The method of claim 71, wherein fusion of the first and the second image is based on mechanically co-registered acquisition, co-registered acquisition supplemented by imaging physics or mutual information based registration.

73. The method of claim 72, further comprising:
using information from a previous image to acquire a subsequent image.

74. The method of claim 72, further comprising:
using information from at least one previous image to optimize the quality of subsequent images.

75. The method of claim 70, wherein the received information comprises;
sensing location and orientation signals of an ultrasound probe relative to a part of an anatomy to be imaged; and
correcting the sensed location and orientation signals of the ultrasound probe relative to a part of an anatomy to be imaged.
